(19) United States
(12) Patent Application Publication
(10) Pub. No.: US 2020/0325505 A1
(43) Pub. Date: Oct. 15, 2020

(54) FILAMENTOUS FUNGI CAPABLE OF PRODUCING VERY LONG CHAIN FATTY ACIDS

(71) Applicants: UT-Battelle, LLC, Oak Ridge, TN (US); University of Tennessee Research Foundation, Knoxville, TN (US)

(72) Inventors: Jessy L. Labbe, Oak Ridge, TN (US); Wellington Muchero, Oak Ridge, TN (US); Timothy J. Tschaplinski, Oak Ridge, TN (US); Matthew R. Entler, Oak Ridge, TN (US)

(21) Appl. No.: 16/381,521

(22) Filed: Apr. 11, 2019

(51) Int. Cl. C12P 7/64; C12N 1/14

(52) U.S. Cl. CPC C12P 7/6409; C12N 1/14

(57) ABSTRACT

Disclosed herein are methods of producing fatty acids haying chains longer than 18 carbon atoms by growing a strain of Mortierella elongata on a fermentation medium comprising a carbon source and a nitrogen source.

### STATEMENT REGARDING FEDERALLY SPONSORED RESEARCH OR DEVELOPMENT

This invention was made with government support under a research project supported by Prime Contract No. DE-AC05-000R22725 awarded by the U.S. Department of Energy. The government has certain rights in this invention.

### BACKGROUND

Very long chain fatty acids (VLCFAs) are generally differentiated from standard fatty acids by a chain length in excess of eighteen carbons. The natural method of synthesis involves the covalent addition of malonyl or acetyl moieties to an existing fatty acid. The precursor fatty acid is typically, though not exclusively, of chain length eighteen or greater due to the high concentration of this length of fatty acid in most cells. The reaction is catalyzed by an elongase enzyme, which may exist in various forms with differing substrate specificities and is generally found only in plants and fungi. Canonical fatty acid lengths are most commonly sixteen or eighteen carbons, and the lengths for the remaining canonical fatty acids are exclusively even-numbered due to the intrinsic functionality of the evolutionarily conserved fatty acid biosynthesis pathway. Elongases permit both longer fatty acid chain lengths and odd-numbered lengths.

The biological purpose of VLCFAs is not clearly understood, although it is hypothesized that plants and fungi utilize VLCFAs for energy storage, signaling, pathogen defense and environmental control.

A subset of VLCFAs are currently utilized in the dietary supplements market, where they are sold as essential fatty acids. Small amounts of similar VLCFAs are also sold to laboratories conducting health-related research.

Interest exists for utilizing VLCFAs to adjust biofuel properties, e.g., flash point or viscosity, as fuel additives due to their atypical lengths. Previous studies have correlated fatty acid chain lengths and saturation degrees to fuel properties of biodiesel blends. For example, increase in the average fatty acid chain length is correlated to a reduction in nitrogen oxide exhaust (Jeevahan et al., Chemical Engineering Communications, 204.10 (2.017): 1202-1223) and less reactive oxide exhausts (Pourkhesalian et al., Environmental science & technology 48.21 (2014): 12577-12585). Currently, VLCFAs are not produced on an industrially relevant scale due to the paucity of organisms which produce them naturally and abundantly.

Mortierella elongata is a filamentous oleaginous fungus with a rapid growth rate and diverse metabolism. Mortierella elongata grows saprotrophically in the soil, or as an endophyte in healthy plant roots. Its quick growth is due in part to coenocytic mycelium that has occasional septa and frequently anastomosing, dichotomously branched hyphae.

### BRIEF SUMMARY

In one aspect, this disclosure provides a method of producing fatty acids having a chain of at least 18 carbon atoms comprising growing a strain of Mortierella elongata on a fermentation medium comprising a carbon source and a nitrogen source.

In some embodiments, the Mortierella elongata strain is selected from the group consisting of Mortierella elongata strain designated as AG77 (ATCC deposit number PTA-125747) and Mortierella elongata strain designated as PMI_93 (ATCC deposit number PTA-125748).

In some embodiments, the carbon source for fermentation comprises at least one carbon source selected from the group consisting of N-Acetyl-D-Glucosamine, L-Aspartic Acid, L-Proline, D-Alanine, D-Trehalose, D-Sorbitol, Glycerol, D-Glucuronic Acid, L-Lactic Acid, D-Glucose-6-Phosphate, D-Galactonic Acid-γ-Lactone, Tween 20, Maltose, D-Aspartic Acid, D-Glucosaminic Acid, 1,2-Propanediol, α-Keto-Glutaric Acid, α-Methyl-D-Galactoside, m-Tartaric Acid, D-Glucose-1-Phosphate, D-Fructose-6-Phosphate, Tween 80, Maltotriose, Adenosine, Glycyl-L-Aspartic Acid, Citric Acid, m-Inositol, D-Threonine, Fumaric Acid, Glycolic Acid, Inosine, Tricarballylic Acid, L-Threonine, Acetoacetic Acid, N-Acetyl-β-D-Mannosamine, Methyl Pyruvate, Tyramine, D-Psicose, Glucuronamide, L-Galactonic Acid-γ-Lactone, Aminoethanol, Mannan, N-Acetyl-Neuraminic Acid, D-Fucose, a-Methyl-D-Glucoside, β-Methyl-D-Galactoside, Palatinose, D-Raffinose, Salicin, D-Glucosamine, D-Lactic Acid Methyl Ester, L-Alaninamide, N-Acetyl-L-Glutamic Acid, L-Arginine, L-Ornithine, L-Phenylalanine, D,L-Carnitine, and Putrescine.

In some embodiments, the nitrogen source for fermentation comprises at least one nitrogen source selected from the group consisting of Nitrate, L-Alanine, L-Glutamic Acid, L-Glutamine, Glycine, L-Histidine, L-Isoleucine, L-Proline, D-Alanine, L-Homoserine, N-Acetyl-D-Glucosamine, N-Acetyl-D-Galactosamine, Cytidine, Parabanic Acid, γ-Amino-N-Butyric Acid, Ala-Asp, Ala-Gin, Ala-Glu, and Ala-Leu.

In some embodiments, the Mortierella elongata is grown under microaerobic conditions. In some embodiments, the Mortierella elongata is grown under anaerobic conditions. In some embodiments, the Mortierella elongata is grown under aerobic conditions.

In some embodiments, the chain fatty acids comprise saturated fatty acids. In some embodiments, the saturated fatty acids are selected from the group consisting of stearic acid, Arachidic acid, Behenic acid, Lignoceric acid, Cerotic acid.

In some embodiments, the fatty acids comprise unsaturated fatty acids. In some embodiments, the unsaturated fatty acids are selected from the group consisting of 11-Eicosenoic acid, Arachidonic acid, Gamma-linolenic acid, Oleic acid, and Linoleic acid.

Another aspect of this invention is directed towards a biofuel additive comprising the fatty acids produced by the method of growing a strain of Mortierella elongata on a fermentation medium comprising a carbon source and a nitrogen source. The term “biofuel additive” refers to compounds or compositions added to a biofuel to improve the biofuel's properties, e.g., burning efficiency or viscosity.

Another aspect of this invention is directed towards a dietary supplement comprising the fatty acids produced by the method of growing a strain of Mortierella elongata on a fermentation medium comprising a carbon source and a nitrogen source. The term “dietary supplement”, also known as “food supplement” or “nutritional supplement”, refers to a preparation intended to supplement the diet and provide nutrients, such as vitamins, minerals, fiber, fatty acids, or amino acids, that may be missing or may not be consumed in sufficient quantities in a person's diet.

### BRIEF DESCRIPTION OF THE FIGURE

FIG. 1. Abundances of fatty acid elongase genes for selected industrially relevant filamentous fungi. Gene counts were obtained from annotated genome assemblies in JGI's MycoCosm database, excluding M. alpina, which was obtained from NCBI's Genome database. M. elongata, Mortierella elongata; M. verticulata, Mortierella verticulata; M. alpina, Mortierella alpina; T. reesei, Trichoderma reesei; S. cerevisiae, Saccharomyces cerevisiae; N. crassa, Neurospora crassa; P. chrysogenum, Penicillium chrysogenum; A. niger, Aspergillus niger.

### DETAILED DESCRIPTION

### Definitions

As used herein, the term “about” refers to a variation within approximately ±10% from a given value.

The term “fatty acid” refers to a carboxylic acid with a long aliphatic chain, which is either saturated or unsaturated. Most naturally occurring fatty acids have an unbranched chain of an even number of carbon atoms, from 4 to 28. Saturated fatty acids have no C═C double bonds. They have the same formula CH₃(CH₂)ₙCOOH, where “n” is an integer equal to or greater than 1. Unsaturated fatty acids have one or more C═C double bonds. The C═C double bonds can give either cis or trans isomers.

As used herein, the phrase “very long chain fatty acid” (VLCFA) refers to a fatty acid that has chains of 18 carbons or longer. In some embodiments, very long chain fatty acids have chains of 18, 20, 22, 24, 26, or 28 carbons.

### General Description

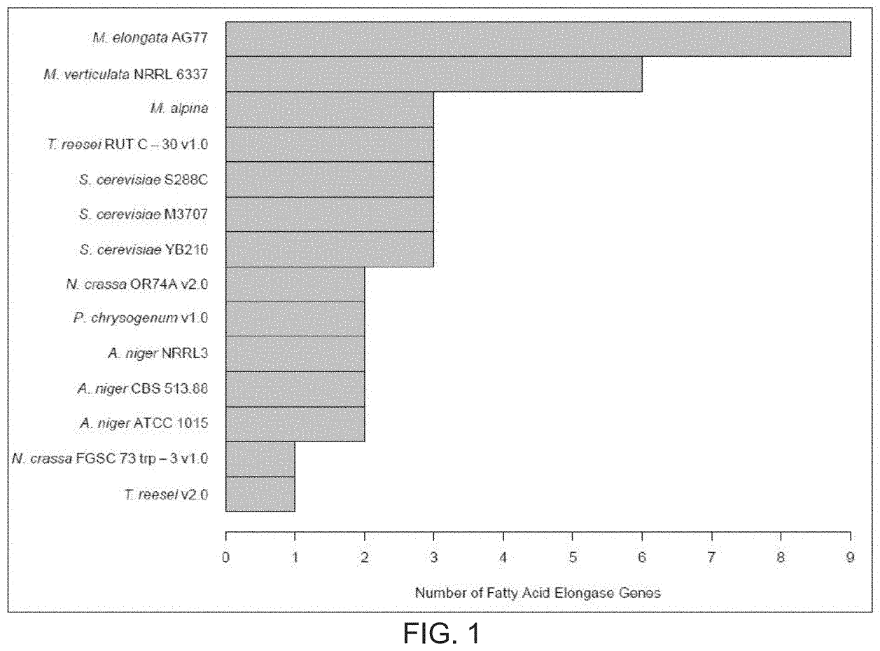

This disclosure is predicated on the discovery that some Mortierella elongata strains contain an increased number of elongase genes compared to other industrially relevant fungi (see FIG. 1), and, therefore, can produce significant amounts of very long chain fatty acids (see Table 2).

In some embodiments, the present disclosure provides a method of producing very long fatty acids using Mortierella elongata strains in a fermentation reaction.

### Very Long Chain Fatty Adds

In some embodiments, very long fatty acids produced by the methods of this disclosure comprise saturated fatty acids.

In some embodiments very long fatty acids of this disclosure are selected from the group consisting of:
Stearic acid with the chemical formula CH₃(CH2)₁₆COOH and the chemical structure

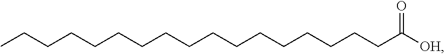 [SMILES: CCCCCCCCCCCCCCCCCC(=O)O]

Arachidic acid with the chemical formula CH₃(CH₂)₁₈COOH and the chemical structure

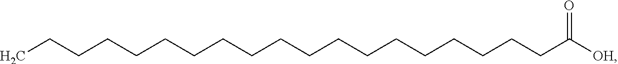 [SMILES: CCCCCCCCCCCCCCCCCCCC(=O)O]

Behenic acid with the chemical formula CH₃(CH₂)₂₀COOH and the chemical structure

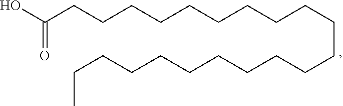 [SMILES: CCCCCCCCCCCCCCCCCCCCCC(=O)O]

Lignoceric acid (aka., tetracosanoic acid) with the chemical formula CH₃(CH₂)₂₂COOH and the chemical structure

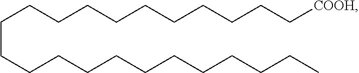 [SMILES: CCCCCCCCCCCCCCCCCCCCCCCC(=O)O]

and
Cerotic acid with the chemical formula CH₃(CH2)₂₄COOH and the chemical structure

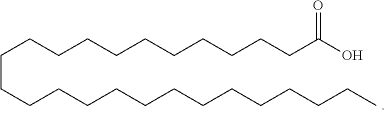 [SMILES: CCCCCCCCCCCCCCCCCCCCCCCCCC(=O)O]

In some embodiments, very long fatty acids produced by the methods of this disclosure comprise unsaturated fatty acids. In some embodiments, the unsaturated very long fatty acids of this disclosure include an unsaturated very long fatty acid selected from the group consisting of: 11-Eicosenoic acid (also called gondoic acid) with the chemical formula C₂₀H₃₈O₂ and the chemical structure

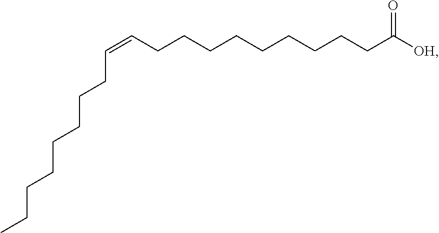 [SMILES: CCCCCCCC/C=C\CCCCCCCCCC(=O)O]

Arachidonic acid with the chemical formula C₂₀H₃₂O₂ and the chemical structure

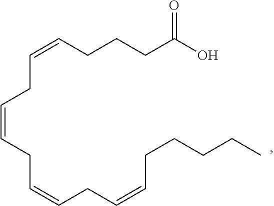 [SMILES: CCCCC/C=C\C/C=C\C/C=C\C/C=C\CCCC(=O)O]

Gamma-linolenic acid with the chemical formula C₁₈H₃₀O₂ and the chemical structure

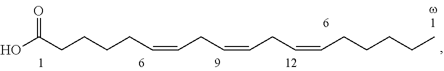 [SMILES: CCCCC/C=C\C/C=C\C/C=C\CCCCC(=O)O]

Oleic acid with the chemical formula C₁₈H₃₄O₂ and the chemical structure

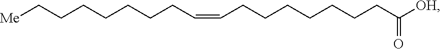 [SMILES: CCCCCCCC/C=C\CCCCCCCC(=O)O]

and
Linoleic acid with the chemical formula C₁₈H₃₂O₂ and the chemical structure

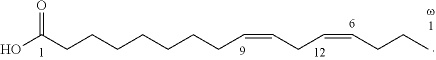 [SMILES: CCC/C=C\C/C=C\CCCCCCCC(=O)O]

### Fermentation Processes for Very Long Fatty Acid Production

In some embodiments, a Mortierella elongata strain is grown under conditions that optimize activity of fatty acid biosynthetic genes and produce the greatest and the most economical yield of fatty acids (e.g., stearic acid, which can in turn increase the production of various omega.-3 and/or .omega.-6 fatty acids). In some embodiments, media conditions that may be optimized include the type and amount of carbon source, the type and amount of nitrogen source, the carbon-to-nitrogen ratio, the oxygen level, growth temperature, pH, length of the biomass production phase, length of the oil accumulation phase and the time of cell harvest.

In some embodiments, a Mortierella elongata strain is grown in a complex medium. In a specific embodiment, the complex medium is yeast extract-peptone-dextrose broth (YPD). In some embodiments, a Mortierella elongata strain is grown in a defined minimal medium that lacks a component necessary for growth and thereby forces selection of the desired expression cassettes. In a specific embodiment, the minimal medium is Yeast Nitrogen Base (DIFCO Laboratories, Detroit, Mich.)).

In some embodiments, the fermentation media comprises a carbon source. In some embodiments, the carbon source includes, but is not limited to, monosaccharides (e.g., glucose, fructose, galactose), disaccharides (e.g., lactose, sucrose, maltose), oligosaccharides, polysaccharides (e.g., glycogen, starch, cellulose or mixtures thereof), sugar alcohols (e.g., glycerol, erythritol, isomalt, lactitol) or mixtures from renewable feedstocks (e.g., cheese whey permeate, cornsteep liquor, sugar beet molasses, barley malt). In some embodiments, carbon sources include alkanes, fatty acids, esters of fatty acids, monoglycerides, diglycerides, triglycerides, phospholipids and various commercial sources of fatty acids including vegetable oils (e.g., soybean oil, corn oil) and animal fats. In some embodiments, the carbon substrate is a one-carbon substrate (i.e., containing a single carbon atom in the molecule). In some embodiments, the one-carbon substrate is carbon dioxide or methanol. In some embodiments, the carbon substrate comprises sugars and/or fatty acids. In some embodiments, the carbon source comprises glucose and/or fatty acids containing between 10 and 22 carbons.

In some embodiments, the carbon source for fermentation comprises at least one substrate selected from the group consisting of N-Acetyl-D-Glucosamine, L-Aspartic Acid, L-Proline, D-Alanine, D-Trehalose, D-Sorbitol, Glycerol, D-Glucuronic Acid, L-Lactic Acid, D-Glucose-6-Phosphate, D-Galactonic Acid-γ-Lactone, Tween 20, Maltose, D-Aspartic Acid, D-Glucosaminic Acid, 1,2-Propanediol, α-Keto-Glutaric Acid, α-Methyl-D-Galactoside, m-Tartaric Acid, D-Glucose-1-Phosphate, D-Fructose-6-Phosphate, Tween 80, Maltotriose, Adenosine, Glycyl-L-Aspartic Acid, Citric Acid, m-Inositol, D-Threonine, Fumaric Acid, Glycolic Acid, Inosine, Tricarballylic Acid, L-Threonine, Acetoacetic Acid, N-Acetyl-β-D-Mannosamine, Methyl Pyruvate, Tyramine, D-Psicose, Glucuronamide, L-Galactonic Acid-γ-Lactone, 2-Aminoethanol, Mannan, N-Acetyl-Neuraminic Acid, D-Fucose, α-Methyl-D-Glucoside, β-Methyl-D-Galactoside, Palatinose, D-Raffinose, Salicin, D-Glucosamine, D-Lactic Acid. Methyl Ester, L-Alaninamide, N-Acetyl-L-Glutamic Acid, L-Arginine, L-Ornithine, L-Phenylalanine, D,L-Carnitine, and Putrescine.

In some embodiments, the fermentation media comprises a nitrogen source. In some embodiments, the nitrogen source is an inorganic source. In a specific embodiment, the inorganic nitrogen source is selected from the group consisting of ammonium sulfate ((NH₄)₂SO₄), urea (CO(NH₂)₂) ammonium nitrate (NH₄NO₃), anhydrous ammonia (NH₃), potassium nitrate (KNO₃), mono-ammonium phosphate (NH₄H₂PO₄), di-ammonium phosphate ((NH₄)₂HPO₄), and Chilean nitrate (NaNO₃).

In some embodiments, the nitrogen source is an organic source. In a specific embodiment, the organic nitrogen source is selected from the group consisting of animal manure, compost, green manure, blood meal, cottonseed meal, feather meal, soybean meal, alfalfa meal, urea and glutamate.

In some embodiments, the nitrogen source for fermentation comprises one or more nitrogen sources selected from the group consisting of Nitrate, L-Alanine, L-Glutamic Acid, L-Glutamine, Glycine, L-Histidine, L-Isoleucine, L-Proline, D-Alanine, L-Homoserine, N-Acetyl-D-Glucosamine, N-Acetyl-D-Galactosamine, Cytidine, Parabanic Acid, γ-Amino-N-Butyric Acid, Ala-Asp, Ala-Gln, Ala-Glu, and Ala-Leu.

In some embodiments, the fermentation medium further contains additives selected from the group consisting of minerals, salts, cofactors, buffers, vitamins, and other components known to those skilled in the art, suitable for the growth of the microorganism and promotion of the enzymatic pathways necessary for polyunsaturated fatty acid (PUFA) production. In some embodiments, the fermentation medium comprises at least one metal ion selected from Mn⁺², Co⁺², Zn⁺², and Mg⁺² that promote synthesis of lipids and PUFAs (Nakahara, T. et al., Ind. Appl. Single Cell Oils, D. J. Kyle and R. Colin, eds. pp 61-97 (1992)).

In some embodiments, the growth medium used in the present disclosure is a commercially-prepared medium. In a specific embodiment, the commercially-prepared medium is Yeast Nitrogen Base from DIFCO Laboratories, Detroit, Mich. In some embodiments, other defined or synthetic growth media are used. Appropriate medium for growth of the particular microorganism will be known by one skilled in the art of microbiology or fermentation science.

In some embodiments, the pH range for the fermentation is between about pH 4.0 to pH 8.0. In some embodiments, the pH range for the fermentation is between pH 5.5 to pH 7.0. In some embodiments, the pH range for the fermentation is between pH 5.8 to pH 6.7. In some embodiments, the pH for the fermentation is about 4.0, 4.1, 4.2, 4.3, 4.4, 4.5, 4.6, 4.7, 4.8, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0,

In some embodiments, the fermentation is conducted under aerobic conditions. The term “aerobic condition” refers to a condition with an oxygen level that is equal to or higher than 21% O₂. In some embodiments, “aerobic condition” refers to 21%, 22%, 23%, 24%, 25%, 26%, 27%, 28%, 29%, 30%, 35% or 40% O₂.

In some embodiments, the fermentation is conducted under anaerobic conditions. The term “anaerobic condition” refers to a condition with an oxygen level that is lower than 1% O₂. In some embodiments, “anaerobic condition” refers to about 0.9%, 0.8%, 0.7%, 0.6%, 0.5%, 0.4%, 0.3%, 0.2%, 0.1% or 0% O₂.

In some embodiments, fermentation is conducted under microaerobic conditions. The term “microaerobic condition” refers to a condition with an oxygen level that is lower than the oxygen level present in the atmosphere, i.e., less than 21% O₂. In some embodiments, “microaerobic condition” refers to 2-10% O₂ levels. In some embodiments, “microaerobic condition” refers to 1%, 2%, 3%, 4%, 5%, 6%, 7%, 8%, 9%, 10%, 15% or 20% O₂.

Various suitable fermentation process designs (e.g., batch, fed-batch or continuous fermentation processes) and considerations for growth are described in WO 2004/101757, which is incorporated herein in its entirety. Solid state fermentation process designs are described in U.S. Pat. No. 6,620,614. Further examples of conditions for fatty acid production are disclosed in U.S. Pat. No. 7,470,532, which is incorporated herein in its entirety.

### Purification of Polyunsaturated Fatty Acids (PUFAs) and Saturated Fatty Acids

In some embodiments, the very long fatty acids produced by the methods of this disclosure comprise polyunsaturated fatty acids (PUFAs).

Fatty acids, including PUFAs and saturated fatty acids, may be found in the host microorganism as free fatty acids or in esterified forms such as acylglycerols, phospholipids, sulfolipids or glycolipids, and may be extracted from the host cell through a variety of means well-known in the art. One review of extraction techniques, quality analysis and acceptability standards for yeast lipids is that of Z. Jacobs (Critical Reviews in Biotechnology 12(5/6):463-491 (1992)). A brief review of downstream processing is also available by A. Singh and O. Ward (Adv. Appl. Microbiol. 45:271-312 (1997)). These references are incorporated herein in entirety.

In some embodiments, means for the purification of PUFAs include extraction with organic solvents, sonication, supercritical fluid extraction (e.g., using carbon dioxide), saponification, and physical means such as presses, or combinations thereof. One is referred to the teachings of WO 2004/101757 for additional details.

### Biofuel Additives

Another aspect of this invention is directed towards a biofuel additive comprising the fatty acids produced by the method of growing a strain of Mortierella elongata on a fermentation medium comprising a carbon source and a nitrogen source. The term “biofuel additive” refers to compounds or compositions added to a biofuel to improve the biofuel's properties, e.g., burning efficiency or viscosity. The term “biofuel” refers to a fuel that is produced through contemporary biological processes, such as agriculture and anaerobic digestion, rather than a fuel produced by geological processes such as those involved in the formation of fossil fuels, such as coal and petroleum, from prehistoric biological matter. Examples of biofuels include ethanol (derived from plant material (e.g., corn or sugarcane) by fermentation), biodiesel (derived from vegetable oils and liquid animal fats), green diesel (derived from algae and other plant sources) and biogas (derived from animal manure and other digested organic material).

### Dietary Supplement Additives

Another aspect of this invention is directed towards a dietary supplement comprising the fatty acids produced by the method of growing a strain of Mortierella elongata on a fermentation medium comprising a carbon source and a nitrogen source. The term “dietary supplement”, also known as “food supplement” or “nutritional supplement”, refers to a preparation intended to supplement the diet and provide nutrients, such as vitamins, minerals, fiber, fatty acids, or amino acids, that may be missing or may not be consumed in sufficient quantities in a person's diet.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The present disclosure is further illustrated by the following non-limiting examples.

### EXAMPLES

### Example 1: Transcript Abundances of Fatty Acid Elongase Genes in Mortierella Elongata 4G77

Transcript counts were obtained from RNA-seq datasets from Mortierella elongata AG77 grown in three carbon sources: kraft lignin, lignocellulose residuals, and glucose.

Data were collected for biological triplicates for each of the three growth conditions. Identified elongases, their average and maximum transcript counts are shown in Table 1. Each elongase is denoted by its JGI (Joint Genome Institute) protein ID number.

TABLE 1
Average number of elongase transcripts
found in Mortierella elongata AG77

| | Average | Maximum |
|---|---|---|
| JGI Protein ID | Transcript Counts | Transcript Counts |
| 14581 | 2 | 7 |
| 132458 | 6 | 15 |
| 140781 | 1 | 3 |
| 165582 | 9 | 18 |
| 1880018 | 7 | 22 |
| 134033 | 1 | 6 |
| 140517 | 5 | 13 |
| 147544 | 5 | 12 |
| 148396 | 3 | 6 |

### Example 2: Detecting Very Long Fatty Acid Concentrations in Mortierella Elongata AG77

### Growth Conditions

Inocula of Mortierella elongata AG77 were isolated from stock culture grown on 20 g/L potato dextrose agar (PDA) using a 10 mm diameter cork borer. Inocula were transferred to a flask containing 100 mL of sterile growth medium comprising either 15 g/L potato dextrose broth (PDB) or 15 g/L PDB and 2 g/L peptone. The liquid cultures were incubated at 20° C. until growth was no longer observed (roughly 5 days). Biomass was separated from the growth medium by straining through a 20 μm nylon basket strainer. While still in the strainer, biomass was rinsed with deionized water. Biomass was transferred into a sterile 50 mL capped conical tube and was flash frozen in liquid nitrogen, lyophilized, and stored at −80° C. until further processing.

### Derivatization

Lyophilized biomass samples were weighed and transferred to glass scintillation vials. Before metabolite extraction, 45 μL of 0.1 g sorbitol in 100 mL water were added to the biomass samples. Extraction was performed by adding 1 mL of 80% (by volume) ethanol/water and capping and incubating at room temperature overnight. The liquid phase was transferred into a new vial by pipette. A 0.5 mL aliquot of the extract was transferred to a new vial and placed under a continuous stream of sterile gaseous nitrogen to evaporate the solvent. After complete evaporation the vial was removed from the nitrogen stream and 0.5 mL of silylation-grade acetonitrile and 0.5 mL of N-methyl-N-trimethylsilyltrifluoroacetamide with 1% trimethylchlorosilane (Thermo Scientific, Bellefonte, Pa.) were added. The vial was heated for 1 h at 70° C. to generate trimethylsilyl derivatives and then stored at room temperature for 2 days to ensure reaction completion.

### Data Collection

A 1 μL aliquot of the trimethylsilyl derivatives solution was injected into an Agilent Technologies Inc. (Santa Clara, Calif.) 5975C inert XL gas chromatograph-mass spectrometer (GC-MS) fitted with a Rtx-5MS with Integra-guard (5% diphenyl/95% dimethyl polysiloxane) 30 m×250 μm×0.25 μm film thickness capillary column. The quadrupole GC-MS was operated in the electron ionization (70 eV) mode with 2.46 full-spectrum (50-650 Da) scans per second; gas (helium) flow was set at 1.33 mL/min with the injection port configured in the splitless mode. The injection port, MS source, and MS quad temperatures were set to 250° C., 230° C., and 150° C., respectively. The initial oven temperature was held at 50° C. for 2 min and was programmed to increase at 20° C./min to 325° C., to hold for 11 min, and then to return to the initial 50° C. hold. Peaks were assigned using a custom mass spectral database, the Wiley Registry 8th Edition mass spectral database, and the NIST 05 mass spectral database.

### Data Analysis

Correction factors were calculated for each VCLFA from external standards by creating an extracted ion chromatogram (EIC) of a single m/z value unique to the VCLFA. The scaling factor was defined as the integrated area under the total ion chromatogram curve divided by the area under the ETC created for sorbitol and each of the VLCFAs previously listed. Concentration of each VLCFA was calculated using the formula cᵢ=C m⁻¹ V Aᵢ cₛMₛ⁻¹Aₛ⁻¹, where cᵢ is concentration of metabolite i, C is the correction factor the metabolite i, m is biomass, V is volume of the extract sample, Aᵢ is integrated area under the ETC curve for metabolite i, cₛ is concentration of sorbitol solution, Mₛ is molar mass of sorbitol, and Aₛ is area under the curve for the sorbitol EIC. Resulting concentrations are in sorbitol equivalents.

### Results

Concentrations were obtained from Mortierella elongata AG77 grown in four unique conditions: Growth condition variables were binary and were defined as presence or absence of excess nitrogen in growth medium, and exposure or lack of exposure to an antibiotic regimen prior to biomass formation and data collection. Mass spectroscopy results are summarized in Table 2 below.

TABLE 2
Very long chain fatty acids detected in
Mortierella elongata by mass spectroscopy.

| | Minimum | Maximum | Number |
|---|---|---|---|
| | Concentration | concentration | Carbon |
| Compound | (mg/g DW) | (mg/g DW) | Atoms |
| 11-eicosenoic acid | 0.03 | 0.11 | 20 |
| arachidic acid | 0.02 | 0.17 | 20 |
| arachidonic acid | 0.30 | 1.80 | 20 |
| gamma-linolenic acid | 0.03 | 0.07 | 18 |
| linoleic acid | 1.04 | 1.80 | 18 |
| monoolein | 0.12 | 0.29 | 21 |
| oleic acid | 3.64 | 4.28 | 18 |
| stearic acid | 0.83 | 4.19 | 18 |
| tetracosanoic acid | 0.08 | 0.40 | 24 |

### Example 3: Elucidation of Carbon and Nitrogen Sources

Two 300 mL volumes of growth medium comprising 1 g/L KH₂PO₄, 1 g/L MgSO₄ 7H₂O, 0.1 g/L CaCl₂, 0.02 g/L FeNaEDTA, 0.1 g/L NaCl, and either 1 g/L NH₄NO₃ or 20 g/L sucrose were prepared. Acidity was adjusted to 5.5 using NaOH and volumes of growth medium were autoclaved 30 min at 121° C. to sterilize. Aliquots of 200 μL of carbon-free medium or nitrogen-free medium were pipetted into each well of a set of 96-well plates previously prepared with lyophilized putative carbon or nitrogen sources (Biolog Inc., Hayward, Calif.). Inocula were prepared by perforating a culture of Mortierella elongata AG77 grown on 20 g/L potato dextrose agar with a 3 mm diameter cork borer and separating the surface mycelial mat from the solid growth medium using tweezers. Each well was inoculated with one inoculum. Cultures were incubated at 21° C. for 24 hours to allow metabolic changes to stabilize, and then 100 μL of 3 g/L tetrazolium chloride in water was added to each well. Formazan formation was observed visually at 2 h, 24 h, and 148 h.

### Results

The carbon sources tested with Mortierella elongata AG77, and results are listed in Table 3 below.

TABLE 3
Growth results on various carbon sources

| Compound | Metabolized | Toxic |
|---|---|---|
| L-Arabinose | no | no |
| N-Acetyl-D-Glucosamine | yes | no |
| D-Saccharic Acid | no | no |
| Succinic Acid | no | no |
| D-Galactose | no | no |
| L-Aspartic Acid | yes | no |
| L-Proline | yes | no |
| D-Alanine | yes | no |
| D-Trehalose | yes | no |
| D-Mannose | no | no |
| Dulcitol | no | no |
| D-Serine | no | no |
| D-Sorbitol | yes | no |
| Glycerol | yes | no |
| L-Fucose | no | no |
| D-Glucuronic Acid | yes | no |
| D-Gluconic Acid | no | no |
| D,L-α-Glycerol- Phosphate | no | no |
| D-Xylose | no | no |
| L-Lactic Acid | yes | no |
| Formic Acid | no | no |
| D-Mannitol | no | no |
| L-Glutamic Acid | no | no |
| D-Glucose-6-Phosphate | yes | no |
| D-Galactonic Acid-γ-Lactone | yes | no |
| D,L-Malic Acid | no | no |
| D-Ribose | no | no |
| Tween 20 | yes | no |
| L-Rhamnose | no | no |
| D-Fructose | no | no |
| Acetic Acid | no | no |
| α-D-Glucose | no | no |
| Maltose | yes | no |
| D-Melibiose | no | no |
| Thymidine | no | no |
| L-Asparagine | no | yes |
| D-Aspartic Acid | yes | no |
| D-Glucosaminic Acid | yes | no |
| 1,2-Propanediol | yes | no |
| Tween 40 | no | no |
| α-Keto-Glutaric Acid | yes | no |
| α-Keto-Butyric Acid | no | no |
| α-Methyl-D-Galactoside | yes | no |
| α-D-Lactose | no | no |
| Lactulose | no | no |
| Sucrose | no | no |
| Uridine | no | no |
| L-Glutamine | no | yes |
| m-Tartaric Acid | yes | no |
| D-Glucose-1-Phosphate | yes | no |
| D-Fructose-6-Phosphate | yes | no |
| Tween 80 | yes | no |
| α-Hydroxy Glutaric Acid-γ-Lactone | no | no |
| α-Hydroxy Butyric Acid | no | no |
| β-Methyl-D-Glucoside | no | no |
| Adonitol | no | no |
| Maltotriose | yes | no |
| 2-Deoxy Adenosine | no | no |
| Adenosine | yes | no |
| Glycyl-L-Aspartic Acid | yes | no |
| Citric Acid | yes | no |
| m-Inositol | yes | no |
| D-Threonine | yes | no |
| Fumaric Acid | yes | no |
| Bromo Succinic Acid | no | no |
| Propionic Acid | no | no |
| Mucic Acid | no | no |
| Glycolic Acid | yes | no |
| Glyoxylic Acid | no | no |
| D-Cellobiose | no | no |
| Inosine | yes | no |
| Glycyl-L-Glutamic Acid | no | no |
| Tricarballylic Acid | yes | no |
| L-Serine | no | no |
| L-Threonine | yes | no |
| L-Alanine | no | no |
| L-Alanyl-Glycine | no | no |
| Acetoacetic Acid | yes | no |
| N-Acetyl-β-D-Mannosamine | yes | no |
| Mono Methyl Succinate | no | no |
| Methyl Pyruvate | yes | no |
| D-Malic Acid | no | no |
| L-Malic Acid | no | no |
| Glycyl-L-Proline | no | no |
| p-Hydroxy Phenyl Acetic Acid | no | no |
| m-Hydroxy Phenyl Acetic Acid | no | no |
| Tyramine | yes | no |
| D-Psicose | yes | no |
| L-Lyxose | no | no |
| Glucuronamide | yes | no |
| Pyruvic Acid | no | no |
| L-Galactonic Acid-γ-Lactone | yes | no |
| D-Galacturonic Acid | no | no |
| Phenylethyl-amine | no | no |
| 2-Aminoethanol | yes | no |
| Chondroitin Sulfate C | no | no |
| α-Cyclodextrin | no | no |
| β-Cyclodextrin | no | no |
| γ-Cyclodextrin | no | no |
| Dextrin | no | no |
| Gelatin | no | no |
| Glycogen | no | no |
| Inulin | no | no |
| Laminarin | no | no |
| Mannan | yes | no |
| Pectin | no | no |
| N-Acetyl-D-Galactosamine | no | no |
| N-Acetyl-Neuraminic Acid | yes | no |
| β-D-Allose | no | no |
| Amygdalin | no | no |
| D-Arabinose | no | no |
| D-Arabitol | no | no |
| L-Arabitol | no | no |
| Arbutin | no | no |
| 2-Deoxy-D-Ribose | no | no |
| i-Erythritol | no | no |
| D-Fucose | yes | no |
| 3-0-β-D-Galacto-pyranosyl-D-Arabinose | no | no |
| Gentiobiose | no | no |
| L-Glucose | no | no |
| Lactitol | no | no |
| D-Melezitose | no | no |
| Maltitol | no | no |
| a-Methyl-D-Glucoside | yes | no |
| β-Methyl-D-Galactoside | yes | no |
| 3-Methyl Glucose | no | no |
| β-Methyl-D-Glucuronic Acid | no | no |
| α-Methyl-D-Mannoside | no | no |
| β-Methyl-D-Xyloside | no | no |
| Palatinose | yes | no |
| D-Raffinose | yes | no |
| Salicin | yes | no |
| Sedoheptulosan | no | no |
| L-Sorbose | no | no |
| Stachyose | no | no |
| D-Tagatose | no | no |
| Turanose | no | no |
| Xylitol | no | no |
| N-Acetyl-D-Glucosaminitol | no | no |
| γ-Amino Butyric Acid | no | no |
| δ-Amino Valeric Acid | no | no |
| Butyric Acid | no | no |
| Capric Acid | no | no |
| Caproic Acid | no | no |
| Citraconic Acid | no | no |
| Citramalic Acid | no | no |
| D-Glucosamine | yes | no |
| 2-Hydroxy Benzoic Acid | no | no |
| 4-Hydroxy Benzoic Acid | no | no |
| β-Hydroxy Butyric Acid | no | no |
| γ-Hydroxy Butyric Acid | no | no |
| α-Keto-Valeric Acid | no | no |
| Itaconic Acid | no | no |
| 5-Keto-D-Gluconic Acid | no | no |
| D-Lactic Acid Methyl Ester | yes | no |
| Malonic Acid | no | no |
| Melibionic Acid | no | no |
| Oxalic Acid | no | no |
| Oxalomalic Acid | no | no |
| Quinic Acid | no | no |
| D-Ribono-1,4-Lactone | no | no |
| Sebacic Acid | no | no |
| Sorbic Acid | no | no |
| Succinamic Acid | no | no |
| D-Tartaric Acid | no | no |
| L-Tartaric Acid | no | no |
| Acetamide | no | no |
| L-Alaninamide | yes | no |
| N-Acetyl-L-Glutamic Acid | yes | no |
| L-Arginine | yes | no |
| Glycine | no | no |
| L-Histidine | no | no |
| L-Homoserine | no | no |
| Hydroxy-L-Proline | no | no |
| L-Isoleucine | no | no |
| L-Leucine | no | no |
| L-Lysine | no | no |
| L-Methionine | no | no |
| L-Ornithine | yes | no |
| L-Phenylalanine | yes | no |
| L-Pyroglutamic Acid | no | no |
| L-Valine | no | no |
| D,L-Carnitine | yes | no |
| Sec-Butylamine | no | no |
| D,L-Octopamine | no | no |
| Putrescine | yes | no |
| Dihydroxy Acetone | no | no |
| 2,3-Butanediol | no | no |
| 2,3-Butanedione | no | no |
| 3-Hydroxy 2-Butanone | no | no |

Conclusions were based on rate and intensity of formazan formation using the 3-4,5-dimethylthiazol-2-yl)-2,5-diphenyltetrazolium bromide (MTT) assay. “Metabolized” indicates more formazan formation than a control containing no carbon source, and “Toxic” indicates less formazan formation than a control containing no carbon source. “Metabolized” and “Toxic” assignments are mutually exclusive. In this example, a compound may be “Metabolized” or “Toxic,” but not both.

Briefly, it was discovered that Mortierella elongata AG77 can metabolize the following carbon sources in production of very long fatty acids: N-Acetyl-D-Glucosamine, L-Aspartic Acid, L-Proline, D-Alanine, D-Trehalose, D-Sorbitol, Glycerol, D-Glucuronic Acid, L-Lactic Acid, D-Glucose-6-Phosphate, D-Galactonic Acid-γ-Lactone, Tween 20, Maltose, D-Aspartic Acid, D-Glucosaminic Acid, 1,2-Propanediol, α-Keto-Glutaric Acid, α-Methyl-D-Galactoside, m-Tartaric Acid, D-Glucose-1-Phosphate, D-Fructose-6-Phosphate, Tween 80, Maltotriose, Adenosine, Glycyl-L-Aspartic Acid, Citric Acid, m-Inositol, D-Threonine, Fumaric Acid, Glycolic Acid, Inosine, Tricarballylic Acid, L-Threonine, Acetoacetic Acid, N-Acetyl-β-D-Mannosamine, Methyl Pyruvate, Tyramine, D-Psicose, Glucuronamide, L-Galactonic Acid-γ-Lactone, 2-Aminoethanol, Mannan, N-Acetyl-Neuraminic Acid, D-Fucose, a-Methyl-D-Glucoside, β-Methyl-D-Galactoside, Palatinose, D-Raffinose, Salicin, D-Glucosamine, D-Lactic Acid Methyl Ester, L-Alaninamide, N-Acetyl-L-Glutamic Acid, L-Arginine, L-Ornithine, L-Phenylalanine, D,L-Carnitine, and Putrescine.

The nitrogen sources used with Mortierella elongata AG77, and results, are listed in Table 4 below.

TABLE 4
Growth results on various nitrogen sources

| Compound | Metabolized | Toxic |
|---|---|---|
| Ammonia | no | no |
| Nitrite | no | no |
| Nitrate | yes | no |
| Urea | no | no |
| Biuret | no | no |
| L-Alanine | yes | no |
| L-Arginine | no | no |
| L-Asparagine | no | no |
| L-Aspartic Acid | no | no |
| L-Cysteine | no | no |
| L-Glutamic Acid | yes | no |
| L-Glutamine | yes | no |
| Glycine | yes | no |
| L-Histidine | yes | no |
| L-Isoleucine | yes | no |
| L-Leucine | no | no |
| L-Lysine | no | no |
| L-Methionine | no | no |
| L-Phenylalanine | no | no |
| L-Proline | yes | no |
| L-Serine | no | no |
| L-Threonine | no | no |
| L-Tryptophan | no | no |
| L-Tyrosine | no | no |
| L-Valine | no | no |
| D-Alanine | yes | no |
| D-Asparagine | no | no |
| D-Aspartic Acid | no | no |
| D-Glutamic Acid | no | no |
| D-Lysine | no | no |
| D-Serine | no | no |
| D-Valine | no | no |
| L-Citrulline | no | no |
| L-Homoserine | yes | no |
| L-Ornithine | no | no |
| 1N-Acetyl-L-Glutamic Acid | no | no |
| N-Phthaloyl-L-Glutamic Acid | no | no |
| L-Pyroglutamic Acid | no | no |
| Hydroxylamine | no | no |
| Methylamine | no | no |
| N-Amylamine | no | no |
| N-Butylamine | no | no |
| Ethylamine | no | no |
| Ethanolamine | no | no |
| Ethylenediamine | no | no |
| Putrescine | no | no |
| Agmatine | no | no |
| Histamine | no | no |
| β-Phenylethyl-amine | no | no |
| Tyramine | no | no |
| Acetamide | no | no |
| Formamide | no | no |
| Glucuronamide | no | no |
| D,L-Lactamide | no | no |
| D-Glucosamine | no | no |
| D-Galactosamine | no | no |
| D-Mannosamine | no | no |
| N-Acetyl-D-Glucosamine | yes | no |
| N-Acetyl-D-Galactosamine | yes | no |
| N-Acetyl-D-Mannosamine | no | no |
| Adenine | no | no |
| Adenosine | no | no |
| Cytidine | yes | no |
| Cytosine | no | no |
| Guanine | no | yes |
| Guanosine | no | no |
| Thymine | no | no |
| Thymidine | no | no |
| Uracil | no | no |
| Uridine | no | no |
| Inosine | no | no |
| Xanthine | no | no |
| Xanthosine | no | no |
| Uric Acid | no | no |
| Alloxan | no | no |
| Allantoin | no | no |
| Parabanic Acid | yes | no |
| D,L-α-Amino-N-Butyric Acid | no | no |
| γ-Amino-N-Butyric Acid | yes | no |
| ε-Amino-N-Caproic Acid | no | no |
| D,L-α-Amino- Caprylic Acid | no | no |
| δ-Amino-N-Valeric Acid | no | no |
| α-Amino-N-Valeric Acid | no | no |
| Ala-Asp | yes | no |
| Ala-Gln | yes | no |
| Ala-Glu | yes | no |
| Ala-Gly | no | no |
| Ala-His | no | no |
| Ala-Leu | yes | no |
| Ala-Thr | no | no |
| Gly-Asn | no | no |
| Gly-Gln | no | no |
| Gly-Glu | no | no |
| Gly-Met | no | no |
| Met-Ala | no | no |

Conclusions were based on rate and intensity of formazan formation using the 3-(4,5-dimethylthiazol-2-yl)-2,5-diphenyltetrazolium bromide (WIT) assay. “Metabolized” indicates more formazan formation than a control containing no nitrogen source, and “Toxic” indicates less formazan formation than a control containing no nitrogen source, “Metabolized” and “Toxic” assignments are mutually exclusive. In this example, a compound may be “Metabolized” or “Toxic,” but not both.

Briefly, it was discovered that Mortierella elongata AG77 can metabolize the following nitrogen sources in production of very long fatty acids: Nitrate, L-Alanine, L-Glutamic Acid, L-Glutamine, Glycine, L-Histidine, L-Isoleucine, L-Proline, D-Alanine, L-Homoserine, N-Acetyl-D-Glucosamine, N-Acetyl-D-Galactosamine, Cytidine, Parabanic Acid, γ-Amino-N-Butyric Acid, Ala-Asp, Ala-Gin, Ala-Glu, and Ala-Leu.

What is claimed is:

1. A method of producing fatty acids having a chain of at least 18 carbon atoms comprising growing a strain of Mortierella elongata on a fermentation medium comprising a carbon source and a nitrogen source.

2. The method of claim 1, wherein the Mortierella elongata strain is selected from the group consisting of Mortierella elongata strain designated as AG77 (ATCC PTA-125747) and Mortierella elongata strain designated as PMI_93 (ATCC PTA-125748).

3. The method of claim 1, wherein the carbon source for fermentation comprises at least one carbon source selected from the group consisting of N-Acetyl-D-Glucosamine, L-Aspartic Acid, L-Proline, D-Alanine, D-Trehalose, D-Sorbitol, Glycerol, D-Glucuronic Acid, L-Lactic Acid, D-Glucose-6-Phosphate, D-Galactonic Acid-γ-Lactone, Tween 20, Maltose, D-Aspartic Acid, D-Glucosaminic Acid, 1,2-Propanediol, α-Keto-Glutaric Acid, α-Methyl-D-Galactoside, m-Tartaric Acid, D-Glucose-1-Phosphate, D-Fructose-6-Phosphate, Tween 80, Maltotriose, Adenosine, Glycyl-L-Aspartic Acid, Citric Acid, m-Inositol, D-Threonine, Fumaric Acid, Glycolic Acid, Inosine, Tricarballylic Acid, L-Threonine, Acetoacetic Acid, N-Acetyl-β-D-Mannosamine, Methyl Pyruvate, Tyramine, D-Psicose, Glucuronamide, L-Galactonic Acid-γ-Lactone, 2-Aminoethanol, Mannan, N-Acetyl-Neuraminic Acid, D-Fucose, α-Methyl-D-Glucoside, β-Methyl-D-Galactoside, Palatinose, D-Raffinose, Salicin, D-Glucosamine, D-Lactic Acid Methyl Ester, L-Alaninamide, N-Acetyl-L-Glutamic Acid, L-Arginine, L-Ornithine, L-Phenylalanine, D,L-Carnitine, and Putrescine.

4. The method of claim 1, wherein the nitrogen source for fermentation comprises at least one nitrogen source selected from the group consisting of Nitrate, L-Alanine, L-Glutamic Acid, L-Glutamine, Glycine, L-Histidine, L-Isoleucine, L-Proline, D-Alanine, L-Homoserine, N-Acetyl-D-Glucosamine, N-Acetyl-D-Galactosamine, Cytidine, Parabanic Acid, γ-Amino-N-Butyric Acid, Ala-Asp, Ala-Gin, Ala-Glu, and Ala-Leu.

5. The method of claim 1, wherein the Mortierella elongata is grown under microaerobic conditions.

6. The method of claim 1, wherein the Mortierella elongata is grown under anaerobic conditions.

7. The method of claim 1, wherein the Mortierella elongata is grown under aerobic conditions.

8. The method of claim 1, wherein the chain fatty acids comprise saturated fatty acids.

9. The method of claim 8, wherein the saturated fatty acids are selected from the group consisting of stearic acid, Arachidic acid, Behenic acid, Lignoceric acid, Cerotic acid.

10. The method of claim 1, wherein the fatty acids comprise unsaturated fatty acids.

11. The method of claim 10, wherein the unsaturated fatty acids are selected from the group consisting of 11-Eicosenoic acid, Arachidonic acid, Gamma-linolenic acid, Oleic acid, and Linoleic acid.

12. A biofuel additive comprising the fatty acids produced by the method of claim 1.

13. A dietary supplement comprising the fatty acids produced by the method of claim 1.
